(19) United States
(12) Patent Application Publication
(10) Pub. No.: US 2010/0280448 A1
(43) Pub. Date: Nov. 4, 2010

(54) ELECTRIC BREAST PUMP

(75) Inventors: Daniel Lantz, Orlando, FL (US); David Littleton, Cotati, CA (US); Anthony Pigliacampo, Menlo Park, CA (US); Ben Chow, San Francisco, CA (US); Anthony Rossetti, San Jose, CA (US)

Correspondence Address: WINSTON & STRAWN LLP;PATENT DEPARTMENT, 1700 K STREET, N.W., WASHINGTON, DC, 20006, US

(73) Assignee: NUK USA LLC

(21) Appl. No.: 12/825,230

(22) Filed: Jun. 28, 2010

Related U.S. Application Data
(62) Division of application No. 11/300,531, filed on Dec. 14, 2005.
(60) Provisional application No. 60/637,709, filed on Dec. 21, 2004.

(51) Int. Cl. A61M 1/06

(52) U.S. Cl. 604/74

(57) ABSTRACT

A breast pump comprises a breast cup and a source of positive and vacuum to the breast cup. The pressure and vacuum are set to an initial value for a fixed period of time, and they are not adjusted by a user. The initial values are selected to allow the breast cup to be repositioned by a user before higher levels of pressure and vacuum are applied.

### CROSS REFERENCE TO RELATED APPLICATIONS

This application is a divisional of U.S. patent application Ser. No. 11/300,531, filed Dec. 14, 2005, which claims priority to U.S. Provisional Patent Application No. 60/637,709, filed Dec. 21, 2004, the disclosure of each of these applications is incorporated herein by reference thereto.

### FIELD OF THE INVENTION

The present invention is generally related to breast pumps for extracting milk from the breasts of human females. More particularly, the present invention is related to such a pump that is designed to massage the breast in order to promote the flow of milk.

### BACKGROUND OF THE INVENTION

For various reasons, a lactating woman may wish to extract and collect her milk, and pumps designed for that purpose are well known. Such a pump typically comprises a cup or funnel that fits over the breast and a neck into which the nipple fits. The other end of the neck is connected to an intake of the actual pumping mechanism, which may be electric or hand operated.

However, the human breast is adapted to respond optimally to suckling by a human baby, whose lips tend to move rhythmically as the baby suckles, massaging the breast. This massaging action stimulates the discharge of milk Merely attempting to suck milk from the nipple is much less effective, because the breast does not respond well.

In the commercial milking of cattle, it has for many years been normal practice for the teat cups of a milking machine to have flexible liners. By cyclically changing the pressure in the space outside the liner, the milking machine massages the teats, encouraging the flow of milk.

U.S. Pat. No. 6,273,868 and U.S. Pat. App. No. 2003/0153869, which are incorporated herein by reference in their entirety, describe breast cups and pumps for human females in which the breast is massaged by supplying a cyclically varying suction and pressure to the nipple tube and to flexible chambers in the breast cup. The supply of pressure to concentric rings of chambers is coordinated to produce a desired pattern of stimulus.

### SUMMARY OF THE INVENTION

One embodiment of the invention provides a breast pump, comprising a breast cup, a source of pressure to the breast cup, and a source of vacuum to the breast cup, wherein the pressure and the vacuum are set to a first pressure and a first vacuum for a set period of time, and both are not changeable by a user during the set period of time.

A further embodiment of the invention provides a method of operating a breast pump comprising applying a first pressure and a first vacuum to a breast cup for a set period of time, wherein the first pressure and the first vacuum are not changeable by a user during the set period of time, and operating the breast pump at an operating pressure and an operating vacuum after the set period of time has elapsed.

After the increase to a working value, the breast pump may operate at a steady value of positive and/or negative pressure that is adjustable in use, for example, is selected by a control operable by the user. In this case, the pressure may increase from the starting value to a fixed first working value, and then ramp from the first working value to the steady value selected by the user. The fixed first working value may then be within the range of values that can be selected for the steady value.

In the case of a breast pump in which positive and/or negative pressure is applied to the breast to stimulate it, and negative pressure is applied to the nipple to suck out milk, both pressures may increase from a starting value to a working value. The increases may take place over the same period of time.

### BRIEF DESCRIPTION OF THE DRAWINGS

For the purpose of illustrating the invention, there are shown in the drawings forms of the invention which are presently preferred; it being understood, however, that this invention is not limited to the precise arrangements and instrumentalities shown.

FIG. 1 is a block diagram of one embodiment of a breast pump according to the invention.

FIG. 2 is a flow chart of the operation of one embodiment of a breast pump according to the invention.

### DETAILED DESCRIPTION

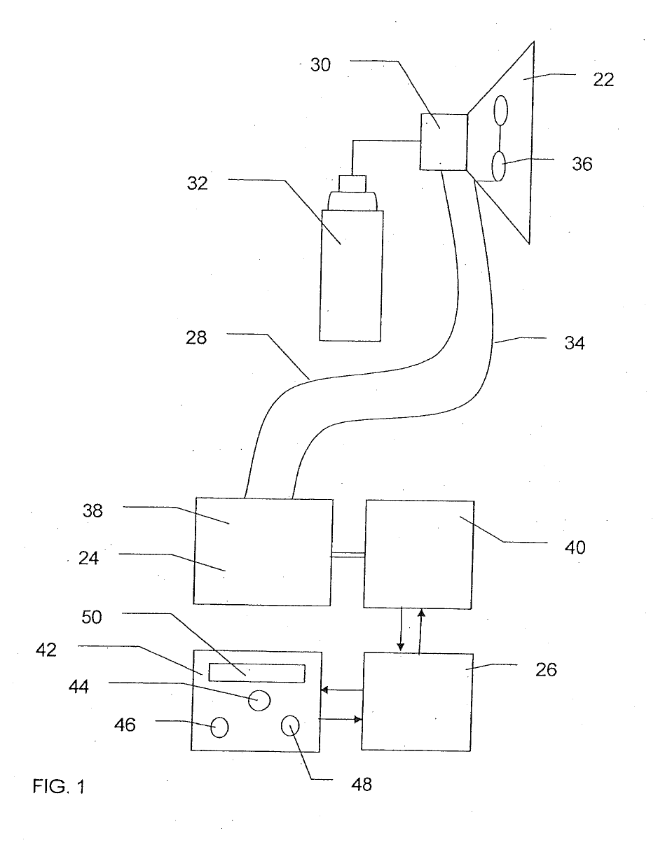

Referring to the accompanying drawings, where like numerals identify like elements, and initially to FIG. 1, one form of breast pump constructed in accordance with an embodiment of the invention, indicated generally by the reference number 20, comprises a breast cup 22, a pump 24, and a pump controller 26.

The breast cup 22 may be similar to those shown in U.S. Pat. No. 6,273,868 and U.S. Pat. App. No. 2003/0153869, in which a first air line 28 supplies negative pressure to a nipple tube 30 of the breast cup 22 in order to suck milk from the nipple into a collecting bottle 32, and at least one second air line 34 supplies air at positive and/or negative pressure to flexible chambers 36 in a conical part of the breast cup 22. In this specification, pressures are described as positive or negative relative to ambient pressure, and are measured from ambient pressure as zero gauge. The chambers 36 may be formed of an elastomeric or other resilient material, so that they can be distorted from a relaxed configuration, and return to the relaxed configuration when the distorting force is removed.

The pump 24 comprises a diaphragm pump 38 and a pump motor 40 that is controllable to vary the pressure, positive and negative, produced by the diaphragm pump 38. In the breast pump 20 shown in FIG. 1, the pressures in the first and second air lines 28 and 34 are produced by a single diaphragm pump 38 driven by a single motor 40. Changes in the output of the diaphragm pump 38 therefore affect the pressures in the lines 28 and 34 proportionately and synchronously.

Depending on the design of the flexible chambers 36, they may be supplied alternately with positive pressure to expand them and negative pressure to contract them. Alternatively, the chambers 36 may be supplied with pressure, for example, positive pressure, intermittently to distort them, and may be restored to a relaxed configuration by the resilience of the material of the chambers 36 when the applied pressure is removed. The applied pressure may be removed by allowing air to flow to or from the surrounding atmosphere through a bleed port. The bleed port may be continuously open, with the diaphragm pump 38 achieving the desired pressure by pumping air within the system faster than it flows through the bleed port.

The pump controller 26 controls the pump motor 40 in accordance with inputs from a control panel 42, which may include an on/off switch 44 and a control 46 to raise and lower at least one pressure level. Other controls 48 to adjust other settings of the pump may also be provided. The control panel 42 may also contain a display 50 with indicators to show the progress of the sequence of steps described below. The controls 46, 48 may be controls such as rotary knobs that show directly the positions to which they are set. Alternatively, the display 50 may also contain indicators to show the levels set by the controls 46 and 48.

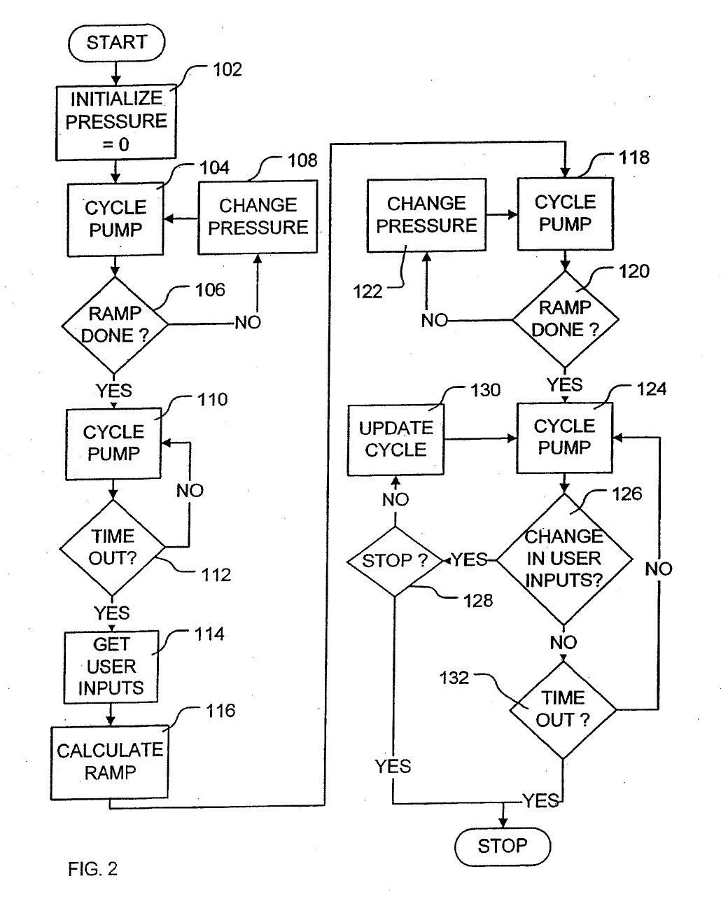

Referring now to FIG. 2, in one embodiment of the breast pump 20, when the pump is initially started and initialized, in step 102 it operates at a factory-set starting pressure. The starting pressure may be too low (that is to say, too close to zero gauge) to be effective in stimulating the release of milk in a typical user, and may be a pressure of approximately zero gauge, that is to say, with the pressure in the lines 28 and 34 substantially the same as the pressure outside.

In steps 104, 106 and 108, the pump operates, while gradually changing the pressure supplied to the line 28 and/or the line 34 in a “ramp” from the starting value to a first working value. If the pressure is starting at or near to zero gauge, the change will be an increase in a positive pressure and/or an increase in the gauge level of a negative pressure. In one embodiment, the most significant factor is the peak pressure during the pump cycle, and a satisfactory ramp can be generated by incrementing the peak pressure for each successive cycle. In this embodiment, in step 104 the pump cycles, in step 106 the controller 26 checks whether the ramp from the starting pressure of step 102 to the first working value is completed, and if the ramp is not completed, in step 108 the pump controller 26 increases the pressure supplied for the next cycle of the pump. If positive and negative pressure are generated on alternate half cycles of the diaphragm pump 38, then the pump controller 26 may repeat steps 104, 106 and 108 for every half cycle. Those skilled in the art will understand how to adapt this process to other types of pump and to other desired ramp profiles.

In step 106, the pump controller 26 may monitor the progress of the pressure change in various ways, for example, by measuring the output pressure in line 28 or line 34, or by counting the number of cycles, or by measuring the time that has elapsed. If the cycle time and the size of the pressure increment are both known, these measures may be substantially equivalent. Those skilled in the art will understand how to monitor the progress of the pressure increase in other ways, or when the cycle time and/or pressure increment is not uniform. In one embodiment, the pump 24 may produce a cycle of pressure every 2 seconds, and the pressure increase may be an increase at a substantially uniform rate over period of about 25 seconds. The ramp from the starting value to the first working value then consists of about 12 steps.

When the pressure has reached the first working value, in steps 110 and 112 the pump controller 26 causes the pump 24 to continue working at that value for a period of, for example, 20 seconds.

In step 114 the pump controller 26 gets from the control panel 42 the pump pressure that the user has set with the control 46. In step 116 the pump controller calculates a ramp from the first working value to the pressure set by the user. In the interests of clarity, steps 114 and 116 are shown in FIG. 2 after the loop at steps 110 and 112. However, steps 114 and 116 may be performed while waiting for the loop to time out at step 112. Alternatively, step 114, and optionally also step 116, could be carried out at an earlier stage. The display 50 may indicate to the user the progress of the breast pump 20 through the process steps shown in FIG. 2. In particular, if the time for which the pump controller 26 dwells at the first working value in steps 110 and 112 is as long as 20 seconds, it may be desirable to indicate this dwell clearly to the user, so the user does not become concerned that the breast pump is not responding to the setting of the pressure control 46.

In steps 118, 120 and 122, the pump controller 26 ramps the pressure from the first working value to the value set by the user. Steps 118, 120 and 122 may use a procedure similar to the procedure used in steps 104, 106 and 108. The first working value may be set near the middle of the range of pressures available to, or typically chosen by, users of the breast pump 20, so the ramp may be a further increase in the pressure or a decrease in the pressure. The ramp may take, for example, 5 seconds. If the pump 24 continues to operate on a 2-second cycle, the ramp then consists of 2 or 3 iterations of step 122. However, most users in practice select pressure values near to the middle of the range, so the pressure change at step 122 is not usually much larger than the change at step 108.

The gradual change of pressure enables the user of the breast pump to adjust the position of the breast cup 22 to the most effective and/or most comfortable position before the breast pump 20 reaches its working level. If the full working pressure were applied at startup, and the breast cup 22 were not correctly positioned, the user could experience some discomfort. With the breast pump 20 operating in accordance with FIG. 2, however, because the pressure level ramps up gradually, any misalignment of the breast cup 22 becomes noticeable, and can be corrected, before it becomes uncomfortable.

Then, in step 124, the pump continues to cycle at a steady value at the pressure set by the user. In step 126, the pump controller may check from time to time whether the user has used the control panel 42 to enter new settings. If so, the new settings are evaluated in step 128. If the new setting is a command to stop the pump, then the pump stops. If the new setting is a change in pressure, then in step 130 the pump controller 26 updates the pump cycle and returns to step 124 to continue pumping at the new pressure. If the new setting is a large change in pressure, the pump controller 26 may go from step 130 to step 122, and use a ramp to change the pressure gradually from the previous setting to the new setting.

If the pump continues to operate, then in step 132 the pump controller 26 may stop the pump after a predetermined time period, for example, one hour.

As an example of pressures used in the breast pump 20, when working in its steady state the pressure may be adjustable by the control 46 between a minimum of zero gauge pressure and a maximum of 240 mm Hg negative gauge pressure in line 28 and 190 mm Hg positive gauge pressure in the line 34. The first working pressure at step 110 may be set between 50 and 150 mm Hg negative gauge pressure in line 28 and between 50 and 110 mm Hg positive gauge pressure in the line 34. The pressures are taken at the peak of the cycle. The minimum of the cycle is substantially zero gauge pressure, produced by allowing the applied pressure to vent to atmosphere. It will be understood that the desired pressure in the line 34 depends on the design of the breast cup 22, and especially on the design of the chambers 36, because the effect of a particular pressure will depend on the size and shape of the chambers, and on how strong a resilient restoring force opposes the pressure.

The present invention may be embodied in other specific forms without departing from the spirit or essential attributes thereof and, accordingly, reference should be made to the appended claims, rather than to the foregoing specification, as indicating the scope of the invention. For example, the pump controller 26 could carry out steps 114 and 116 as part of the initialization procedure in step 102, and then ramp the pressure directly from the starting value to the value set by the user.

If the pressure is ramped in two stages from the starting value to a first working value and then to the steady value set by the user, the pause at the first working value in steps 110 and 112 is not necessary. However, this pause is useful to allow time for steps 114 and 116, and avoids an abrupt transition if the ramps before and after the pause are in opposite directions.

Although ramps having a constant rate of change of pressure over time are simple to implement, those skilled in the art will understand how to adapt the embodiment described to produce a varying rate of change of pressure if desired, for example, to blend more smoothly into the steady pressure of steps 110 and 124.

Although a fixed time for the ramp in steps 118, 120 and 122 has been described, the ramp could be, for example, a fixed rate of change, with the time taken depending on the amount of the change, or some compromise between constant time and constant rate.

Although a pump 24 with a fixed cycle time has been described, the pump could have a user-selected cycle time set by the control 48. In that case, the user-selected cycle time could be applied from the beginning, or it could be obtained at step 114 and applied during the ramp loop of steps 118, 120 and 122.

Although the breast pump 20 has been shown in FIG. 1 with a single breast cup 22, it may alternatively be provided with two breast cups 22, for simultaneous extraction of milk from both breasts of a human female. Positive and negative pressure may be supplied to both breast cups by T connectors in the lines 28 and 34. The breast pump 20 may be designed to operate with either a single breast cup or two breast cups. The maximum pressure available may then be lower when two breast cups are used than when a single breast cup is used.

Other variations are contemplated and will be understood by those in the art.

What is claimed is:

1. A method of operating a milk breast pump comprising:
a controllable pump, and
a breast cup including a nipple tube connected to a first air line and flexible chambers connected to a second air line,
said controllable pump supplying negative pressure to said first air line and actuating positive and/or negative pressure to said second air line whereby changes in operation of said controllable pump affect the pressures in said first and second air lines proportionately and synchronously,
said method comprising the steps of
automatically ramping up the starting operation of said controllable pump from a low starting value to a predetermined first working value for gradually changing the pressures supplied to said first and second air lines, such a gradual ramping up allowing a user of said breast cup to adapt comfortably said breast cup on a breast during said ramping up;
stabilizing the operation of said controllable pump at said first working value for a set period of time whereby the pressures corresponding to said first working value cannot be changed during said set period of time; and
operating up said controllable pump at the end of said set period of time from said first working value to a second working value selected by said user.

2. The method according to claim 1, wherein operating up said controllable pump after said set period of time is a ramping up.

3. The method according to claim 1, wherein the first working value of said operable pump provides negative gauge pressure, set between 50 and 150 mm Hg in said first air line.

4. The method according to claim 1, wherein the first working value of said operable pump provides positive gauge pressure, set between 50 and 110 mm Hg, in said second air line.

5. The method according to claim 1, wherein said set period of time is at least 10 seconds.

6. The method according to claim 1, wherein said set period of time is at least 20 seconds.

7. The method according to claim 1, wherein said controllable pump is operated at fixed cycle time to produce a constant cycle of pressure.

8. The method according to claim 7, wherein said cycle of pressure is every 2 seconds.

9. A milk breast pump comprising:
a controllable pump,
a breast cup including a nipple tube connected to a first air line and flexible chambers connected to a second air line,
said controllable pump supplying negative pressure to said first air line and actuating positive and/or negative pressure to said second air line whereby changes in operation of said controllable pump affect pressure in said first and second air line proportionately and synchronously, and
control means for automatically ramping up the operation of said controllable pump from a starting value to a predetermined first working value for gradually changing the pressures supplied to said first and second air lines whereby said gradual ramping up allows a user of said breast cup to adapt comfortably said breast cup on a breast during said ramping up,
said control means being further arranged to stabilize the operation of said controllable pump at said first working value for a set period of time during which the pressures corresponding to said first value of said controllable pump cannot be changed and to enable operating up of said controllable pump from said first working value to a second working value above said first working value, said second working value being chosen by said user.

10. The milk breast pump according to claim 9, wherein said control means are arranged for ramping up said controllable pump after said set period of time.

11. The milk breast pump according to claim 10, wherein the first working value of said operable pump provides negative gauge pressure, set between 50 and 150 mm Hg in said first air line.

12. The milk breast pump according to claim 10, wherein the first working value of said operable pump provides positive gauge pressure, set between 50 and 110 mm Hg, in said second air line.

13. The milk breast pump according to claim 10, wherein said set period of time is at least 10 seconds.

14. The milk breast pump according to claim 10, wherein said set period of time is at least 20 seconds.

15. The milk breast pump according to claim 10, wherein said controllable pump is operated at fixed cycle time to produce a constant cycle of pressure.

16. The milk breast pump according to claim 15, wherein said cycle of pressure is every 2 seconds.
